(19) United States
(12) Patent Application Publication
(10) Pub. No.: US 2023/0055843 A1
(43) Pub. Date: Feb. 23, 2023

(54) SYSTEMS, METHODS, AND APPARATUSES TO PROVIDE CHEST TUBE INSERTION FEEDBACK

(71) Applicant: The Board of Regents of the University of Texas System, Austin, TX (US)

(72) Inventor: Caroline Park, Dallas, TX (US)

(21) Appl. No.: 17/889,946

(22) Filed: Aug. 17, 2022

Related U.S. Application Data
(60) Provisional application No. 63/234,495, filed on Aug. 18, 2021.

(51) Int. Cl. A61B 5/00; G01L 1/20; G09B 23/28; G08B 21/18

(52) U.S. Cl. CPC A61B 5/0053; G01L 1/20; G09B 23/285; G08B 21/182

(57) ABSTRACT

A chest tube insertion test device for performing a chest tube insertion training procedure at least partially encloses an insertion tool and includes a microcontroller communicatively coupled to one or more force-sensing resistors. The one or more force-sensing resistors convert an insertion force into a voltage signal sent to the microcontroller. A feedback generator compares the chest tube insertion force to one or more predetermined threshold values and provides feedback to a trainee. The feedback can include an instruction to increase, decrease, or maintain the chest tube insertion force. The feedback is one or more audio cues and/or one or more visual cues (e.g., represented by audio files and/or visual data files stored at the chest tube insertion test device). The chest tube insertion test device detects an insertion force rate of change indicating that the insertion tool has passed through an inner pad of the training model.

### CROSS-REFERENCE TO RELATED APPLICATION

This application claims priority under 35 U.S.C. § 119 to Application No. 63/234,495, filed Aug. 18, 2021 and entitled “SYSTEMS, METHODS, AND APPARATUSES TO PROVIDE CHEST TUBE INSERTION FEEDBACK,” which is incorporated in its entirety herein by reference.

### BACKGROUND

### 1. Field

The present disclosure relates generally to systems, methods, and apparatuses to provide chest tube insertion feedback. In at least one example, the present disclosure relates to a chest tube insertion test device configured to generate audio cues and visual cues during a training procedure.

### 2. Discussion of Related Art

A chest tube insertion procedure (i.e., a thoracostomy) is often performed to remove fluid or air from the pleural space of a patient. It is a difficult medical procedure with little room for error because the chest tube must be inserted to a precise depth to reach the pleural space. During the chest tube insertion procedure, medical personnel must pierce the chest of the patient with an insertion tool and insert a hollow tube directly into the chest between the ribs. An amount of force is applied with the insertion tool to push the chest tube into the proper placement while avoiding pushing the chest tube beyond the desired depth (e.g., into the diaphragm of the patient).

Due to the difficult nature of the chest tube insertion procedure, and the negative patient outcomes that result from improper chest tube placement, medical personnel typically undergo training for the procedure on a mannequin. However, the effectiveness of the training is limited because it is difficult for medical personnel to assess an amount of force being applied to the mannequin. Accordingly, less experienced medical personnel may lack confidence when performing the chest tube insertion procedure and may be prone to errors, even after undergoing training.

It is with these observations in mind, among others, that various aspects of the present disclosure were conceived and developed.

### BRIEF SUMMARY

The presently disclosed technology address the foregoing problems by providing chest tube insertion feedback during a training procedure. A chest tube insertion test device or apparatus generates the feedback based on an insertion force detected by one or more force-sensing resistors. The feedback indicates how much force a trainee is applying and provides real-time instructions indicating whether the amount of force should be increased, decreased, or maintained. The chest tube insertion test device or apparatus detects when the insertion tool is at the proper insertion depth and alerts the trainee that the proper insertion depth has been reached. By providing real-time, force-related feedback during the chest tube insertion training procedure, the systems disclosed herein significantly improve the training procedure.

In some examples, a device or apparatus configured to provide feedback for a chest tube insertion can include a case coupled to an insertion tool; one or more force-sensing resistors coupled to the case; one or more feedback output devices; a microcontroller communicatively coupled to the one or more force-sensing resistors and the one or more feedback output devices; and/or one or more memory devices configured to store instructions that, when executed by the microcontroller, cause the apparatus or device to generate, using the one or more force-sensing resistors, an input signal representing a chest tube insertion force, determine whether the chest tube insertion force exceeds a predetermined threshold value, and/or output, using the one or more feedback output devices, the feedback indicating whether the chest tube insertion force exceeds the predetermined threshold value.

In some instances, the one or more force-sensing resistors are a plurality of force-sensing resistors electrically wired in parallel. The predetermined threshold value can correspond to an expected force range, and/or the feedback can include an indication of whether the chest tube insertion force is within the expected force range. Furthermore, the instructions, when executed by the microcontroller, can further cause the apparatus to determine the chest tube insertion force is less than the predetermined threshold value, and/or the feedback can include an alert instructing a user to push the insertion tool with more force. The instructions, when executed by the microcontroller, can further cause the apparatus to determine the chest tube insertion force exceeds the predetermined threshold value, and the feedback can include a first alert instructing a user to push the insertion tool with less force and/or a second alert instructing the user to inspect an insertion area.

In some instances, the one or more feedback output devices include at least one of a display or an audio speaker, the first alert includes at least one of a first visual cue presented on the display or a first audio file outputted by the audio speaker, and the second alert includes at least one of a second visual cue presented on the display and a second audio file outputted by the audio speaker. The chest tube insertion force can correspond to between zero kilograms and five kilograms, or 15-20 newtons. The case can be removably secured to the insertion tool, and the case, when secured to the insertion tool, can at least partially enclose a portion of the insertion tool. Moreover, the insertion tool can be a Kelly clamp tool, and the portion of the insertion tool at least partially enclosed by the case can be a handle portion of the Kelly clamp tool. The apparatus can further include a housing coupled to the case, wherein the microcontroller is at least partially contained in the housing, and the one or more feedback output devices are coupled to the housing. In some instances, the apparatus further includes a power supply at least partially contained in the housing, the power supply providing power to the microcontroller.

In some examples, a system to provide feedback for a chest tube insertion includes a force sensor coupled to an insertion tool; a feedback output device; a microcontroller communicatively coupled to the force sensor and/or the feedback output device; and/or one or more memory devices storing instructions that, when executed by the microcontroller, cause the system to: receive, using the force sensor, an input signal representing a chest tube insertion force, and/or generate, using the feedback output device, an output indicating that: the chest tube insertion force is below a predetermined threshold range, the chest tube insertion force is within the predetermined threshold range, and/or the chest tube insertion force is greater than the predetermined threshold range.

In some instances, the feedback output device includes an audio speaker, and/or generating the output includes playing an audio file with the audio speaker. The feedback output device can include a display, and/or generating the output includes presenting one or more visual cues on the display. Additionally, the chest tube insertion force can be a first chest tube insertion force, and the output can further include an instruction to: push the insertion tool with a second chest tube insertion force being less than the first chest tube insertion force, push the insertion tool with the second chest tube insertion force being greater than the first chest tube insertion force, or continue pushing the insertion tool with the first chest tube insertion force.

In some examples, a method to provide feedback for a chest tube insertion can comprise: generating, with a force sensor coupled to an insertion tool, an input signal representing a chest tube insertion force for the insertion tool; receiving the input signal at a microcontroller communicatively coupled to the force sensor; determining whether the chest tube insertion force exceeds a predetermined threshold value; and/or outputting, at one or more feedback output devices, the feedback indicating whether the chest tube insertion force exceeds the predetermined threshold value.

In some instances, the force sensor can include one or more force-sensing resistors integrated into a case at least partially enclosing the insertion tool. Generating the input signal can include: inserting the insertion tool into a training model, and/or causing an inner pad of the training model to be pressed against the force sensor. The method can further include: detecting, with the force sensor, a change in force caused by the insertion tool passing through the inner pad of the training model. Additionally, the feedback can further indicate, in response to the change in force, that a training procedure is complete.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of apparatuses, systems, and methods consistent with the disclosed subject matter and, together with the description, serves to explain advantages and principles consistent with the disclosed subject matter, in which:

FIG. 1 illustrates an example system including a chest tube insertion test device for providing feedback during a chest tube insertion training procedure;

FIGS. 2A and 2B illustrate an example system including a chest tube insertion test device including a case, which can form at least a portion of the system of FIG. 1;

FIG. 3 illustrates an example system including various components of a chest tube insertion test device, which can form at least a portion of the system of FIG. 1;

FIG. 4 illustrates an example system including electrical circuitry of a chest tube insertion test device, which can form at least a portion of the system of FIG. 1;

FIGS. 5A and 5B illustrate an example system including a housing of a chest tube insertion test device, which can form at least a portion of the system of FIG. 1;

FIG. 6 illustrates an example method for testing and configuring a chest tube insertion test device, which can be performed by the system of FIG. 1;

FIG. 7 illustrates an example method for providing feedback with a chest tube insertion test device, which can be performed by the system of FIG. 1; and

FIG. 8 illustrates an example method for performing a chest tube insertion training procedure with a chest tube insertion test device, which can be performed by the system of FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

### II. General Architecture

The systems disclosed herein improve a training procedure for chest tube insertions by providing force-related feedback with a chest tube insertion test apparatus or device. The chest tube insertion test device can include a case housing one or more force-sensing resistors for converting a force with which an insertion tool is pushed into a training model (e.g., a “chest tube insertion force”) into a voltage signal. A microcontroller of the chest tube insertion test device receives and analyzes the voltage signal to determine which feedback to output to the trainee. For instance, the microcontroller can compare the chest tube insertion force to various stored threshold values to determine whether the chest tube insertion force is below, within, or above an expected force range. The expected force range represents an appropriate amount of force for properly performing the chest tube insertion procedure. The chest tube insertion test device outputs audio cues and/or visual cues instructing the trainee based on how the applied force compares to the threshold values. Moreover, the microcontroller can detect the occurrence of a sudden drop in force indicating that the insertion tool has reached the end of an inner pad inside the training model, which corresponds to reaching a target insertion depth for the chest tube (e.g., that the insertion tool has entered the thoracic cavity). For instance, the training model can include replaceable inner pads having a similar resistance as human tissue to simulate the resistance of human tissue. Upon reaching the end of the pad, the resistance drops to zero causing a sudden drop in the measured force, which triggers output of an audio cue and/or a visual cue indicating to the trainee that the insertion tool has reached its target insertion depth.

The chest tube insertion test device can attach to (e.g., partially enclose) an insertion tool, such as a Kelly clamp tool, that the trainee would actually use during a real chest tube insertion procedure. As such the chest tube insertion test device improves the training procedure by providing real-time feedback indicating how much force the trainee is applying and whether the amount of force should be increased, decreased, or maintained. The chest tube insertion test device detects when the insertion tool is at the proper insertion depth and alerts the trainee that the proper insertion depth has been reached. By providing real-time, force-related feedback during the chest tube insertion training procedure, the systems disclosed herein significantly improve the training procedure. The trainee becomes more familiar with the proper amount of force to be applied and the proper depth to reach with the insertion tool. Because the insertion tool is the same insertion tool used in real world scenarios, using the chest tube insertion test device increases a confidence level and a competence level of the trainee for performing chest tube insertions. As such, errors for chest tube insertions are decreased and patient outcomes are improved. Additional advantages of the systems discussed herein will become apparent from the detailed description below.

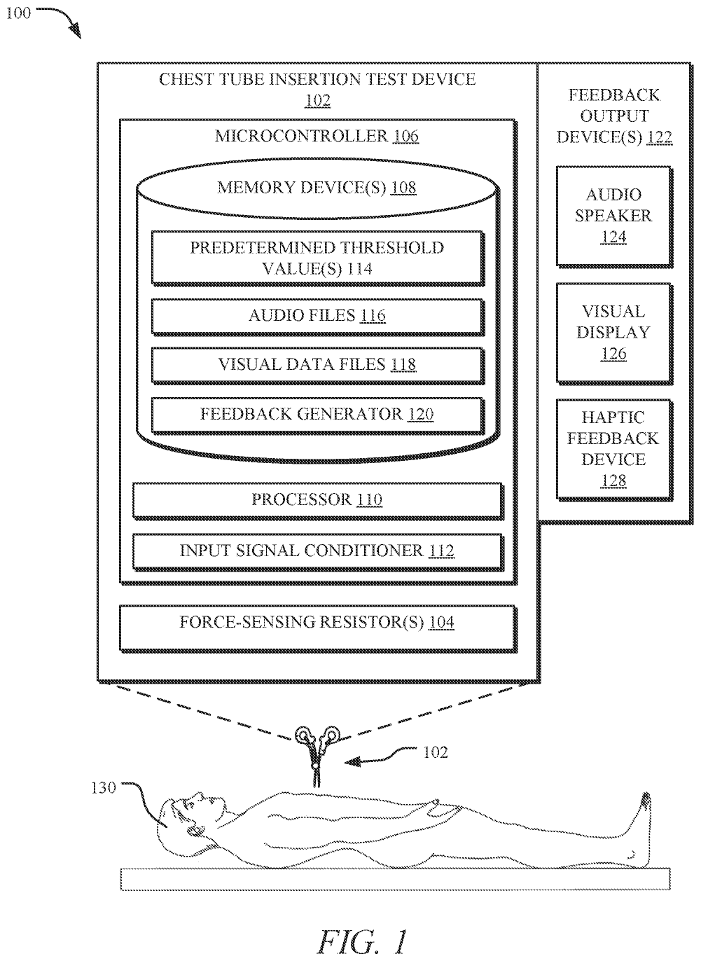

FIG. 1 illustrates an example system 100 for providing feedback for a chest tube insertion. The system 100 can include a chest tube insertion test device 102 for detecting a force or pressure with which the chest tube insertion test device 102 is pushed into a training model, for instance, during a training procedure. The chest tube insertion test device 102 can include one or more force-sensing resistor(s) 104 for converting a force or pressure into a voltage signal, and a microcontroller 106 to receive the voltage signal and generate the feedback based on the voltage signal. The feedback can indicate to a trainee whether the amount of force being applied with the chest tube insertion test device 102 should be increased, decreased, maintained, or if other actions should be taken.

In some examples, the microcontroller 106 includes one or more memory device(s) 108 storing executable instructions (e.g., software and/or algorithm modules) that, when executed by a processor 110 of the microcontroller 106, cause the chest tube insertion test device 102 to perform the operations discussed herein. The chest tube insertion test device 102 can further include an input signal conditioner 112 for converting unprocessed, analogue voltage signals generated by the one or more force-sensing resistor(s) 104 into a processed, normalized, and/or digitized voltage signal to be analyzed by the microcontroller 106. The input signal conditioner 112 can be a hardware or software component separate from the microcontroller and/or the input signal conditioner 112 can form a part of the microcontroller 106 (e.g., as a set of computer-readable instructions that normalize the voltage signal generated by the force-sensing resistor(s) 104). The microcontroller 106 can be an Arduino Uno board and can have 7-volt to 12-volt input ports and a 5-volt output port.

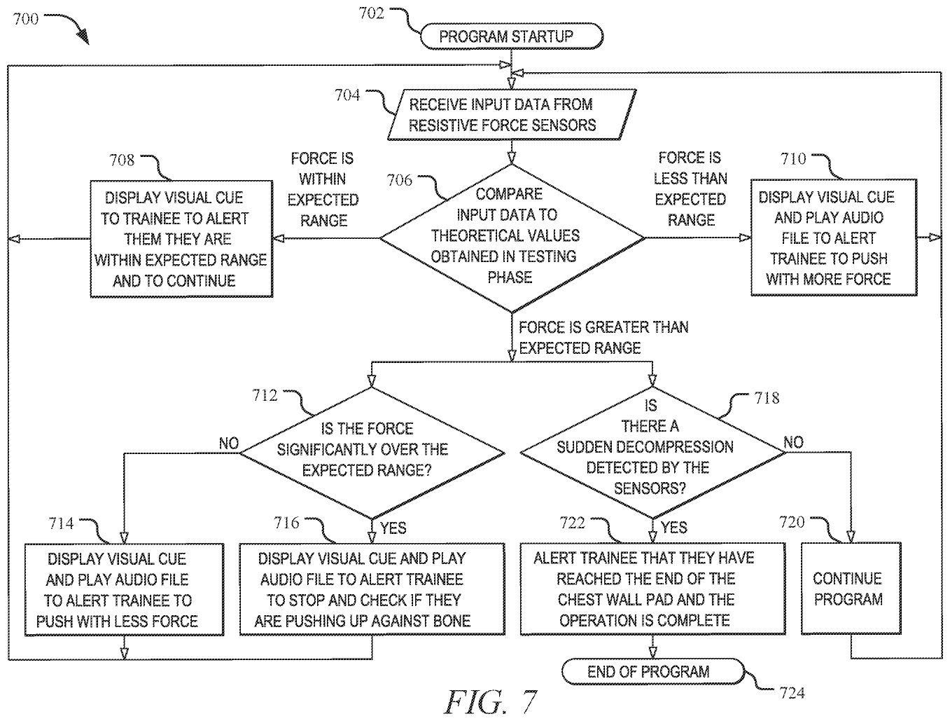

In some examples, the one or more memory device(s) 108 store various predetermined threshold values 114, audio files 116, visual data files 118, and a feedback generator 120. The feedback generator 120, in some instances, analyzes the voltage signal originating from the one or more force-sensing resistor(s) 104 using the predetermined threshold value(s) 114 and determines which feedback to output. The feedback can be any of the audio files 116 or the visual data files 118. For instance, the feedback generator 120 can determine to output a particular audio file of the one or more audio file(s) 116 or a particular visual data file of the one or more visual data file(s) 118 in response to the voltage signal (e.g., a normalized or processed voltage signal) being above or below the one or more predetermined threshold value(s) 114. Operations performed by the feedback generator 120 to generate the feedback are discussed in greater detail below regarding FIG. 7.

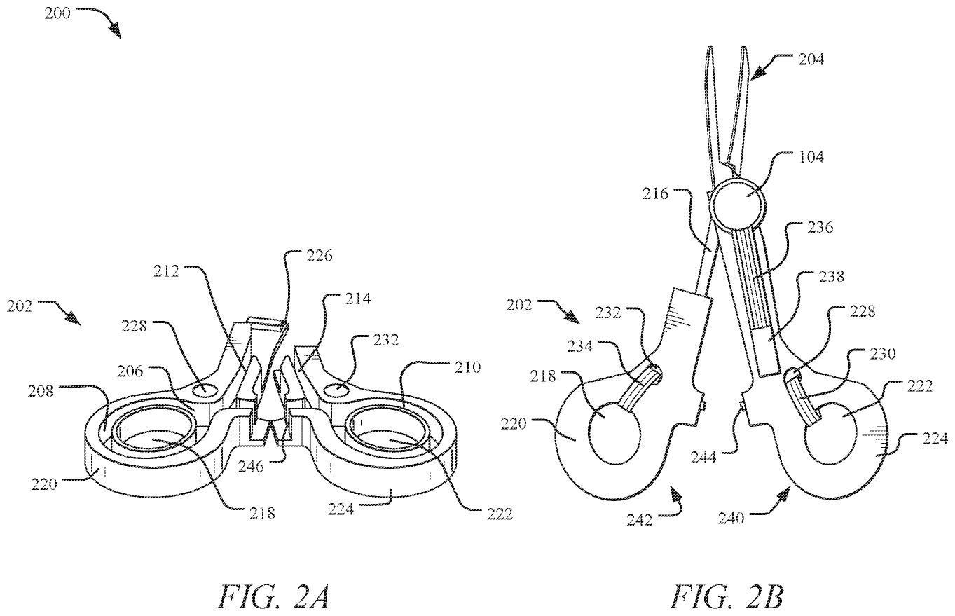

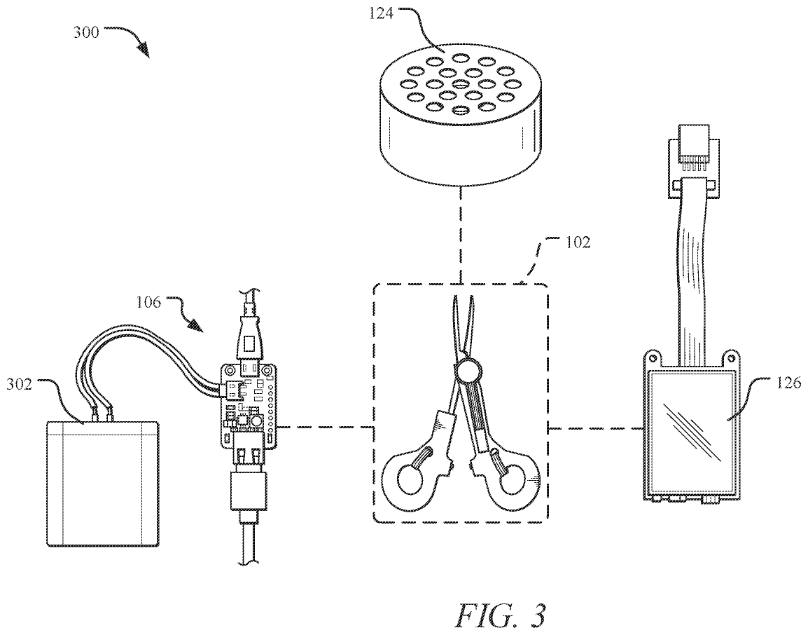

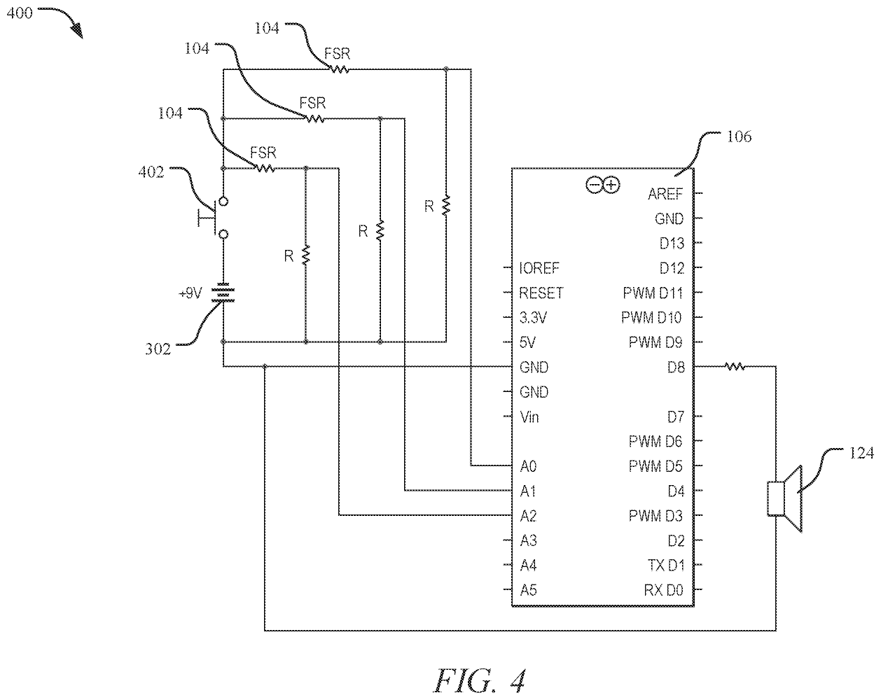

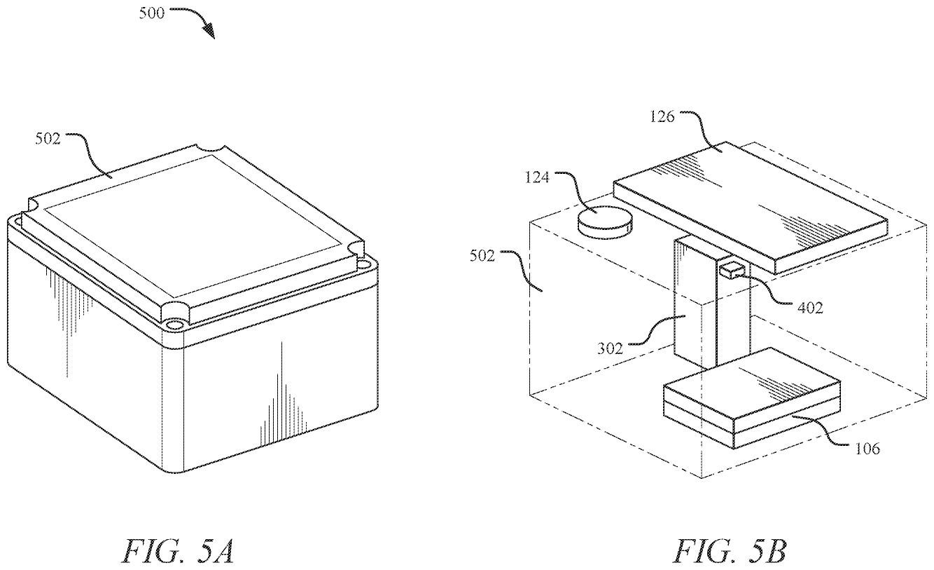

In some examples, the chest tube insertion test device 102 outputs the feedback using one or more feedback output device(s) 122. The one or more feedback output device(s) 122 can include an audio speaker 124, a visual display 126, and/or a haptic feedback device 128. The feedback generator 120 can select the audio file 116 to output via the audio speaker 124 (e.g., as a voice message, an alarm sound combinations thereof, etc.). The feedback generator 120 can select the visual data file 118 to output via the visual display 126 (e.g., as a display of text, an instructional image, an instructional video, combinations thereof, etc.). The feedback generator 120 can select a haptic feedback file to output via the haptic feedback device (e.g., causing the chest tube insertion test device 102 to move, vibrate, or present a braille message). In some instances, the feedback includes audio cue beeps and a color-coded barometer (e.g., red-to-green). Moreover, the chest tube insertion test device 102 can include a case (e.g., case 202 in FIG. 2), a power supply (e.g., power supply 302 in FIG. 3), internal circuitry (e.g. FIG. 4) and/or a housing (e.g., housing 502 in FIG. 5), as discussed below. The system 100 can further include a training model 130 (e.g., a training mannequin) for receiving an insertion of the chest tube insertion test device 102 during the training procedure.

In some examples, the one or more memory device(s) 108 include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The data storage device(s) may include volatile memory (e.g., dynamic random access memory (DRAM), static random access memory (SRAM), etc.) and/or non-volatile memory (e.g., read-only memory (ROM), flash memory, etc.). The data storage device may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions. machine-readable media. It will be appreciated that the machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures. The machine-readable media may store instructions that, when executed by the processor 110, cause the systems to perform the operations disclosed herein.

In some examples, the processor 110 can include a central processing unit (CPU), a microprocessor, a digital signal processor (DSP), and/or one or more internal levels of cache. The processor 110 can be or form a portion of the microcontroller 106. There may be one or more processors 110, such that the processor 110 comprises a single central-processing unit, or a plurality of processing units capable of executing instructions and performing operations in parallel with each other.

FIGS. 2A and 2B illustrate an example system 200 including a case 202 of the chest tube insertion test device 102 for attaching components of the chest tube insertion test device 102 to an insertion tool 204 (e.g., by at least partially enclosing the insertion tool 204). For instance, the case 202 can snap onto the insertion tool 204 as a retrofit of the insertion tool 204 to form the chest tube insertion test device 102. Alternatively, the chest tube insertion test device 102 and the insertion tool 204 can together form a stand-alone or fully-integrated tool manufactured as a single component specifically designed for chest tube insertion testing.

In some examples, the chest tube insertion test device 102 is a retrofit of the insertion tool 204. For instance, the case 202 can have various contours, channels, profiles, and/or shapes that correspond to the contours and shapes of the insertion tool 204, such that the insertion tool 204 easily snaps into the case 202. The case 202 can include a body formed of a rigid material (e.g., plastic, carbon fiber, metal, composites, and the like) with one or more channels 206 formed into the case 202 for receiving the insertion tool 204. For instance, a first loop channel 208 of the one or more channels 206 can receive a first handle loop of the insertion tool 204, and a second loop channel 210 of the one or more channels 206 can receive a second handle loop of the insertion tool 204. A first shaft channel 212 can extend from the first loop channel 208 for receiving a first shaft of the insertion tool 204, and a second shaft channel 214 can extend from the second loop channel 210 for receiving a second shaft 216 of the insertion tool 204. The case 202 can include a first loop opening 218 defined by a first gripping loop 220 and a second loop opening 222 defined by a second gripping loop 224. The first gripping loop 220 and the second gripping loop 224 are operable to receive a first finger and a second finger, respectively, of the trainee.

In some examples, the case 202 includes a terminating end 226 of the first shaft channel 212 opposite from the first loop channel 208. The force-sensing resistor(s) 104 can mount or couple to the case 202 at the terminating end 226. The case 202 can further include a first attachment opening 228 which receives a first attachment ring 230 and a second attachment opening 232 which receives a second attachment ring 234. The first attachment ring 230 and the second attachment ring 234 are operable to secure the case 202 in place around the insertion tool 204. The first attachment ring 230 can pass through the first attachment opening 228 and the second attachment ring 234 can pass through the second attachment opening 232, such that the first attachment ring 230 and the second attachment ring 234 wrap around both the case 202 and the insertion tool 204. Additionally or alternatively, the case 202 can be fixed to the insertion tool 204 with a friction-fit, an adhesive, a hook-and-loop arrangement, and the like.

In some examples, the force-sensing resistor(s) 104 can couple to the case 202 at the terminating end 226. One or more wires 236 can extend from the force-sensing resistor(s) 104 along a portion of the case 202 (e.g., above the first shaft channel 212) to a wire connector 238 (e.g., at the microcontroller 106). The case 202 can include multiple, separate case portions that are movable with respect to each other, so that the insertion tool 204 has mostly unobstructed movement when nested in the case 202. For instance, the insertion tool 204 can be a Kelly clamp tool and a first case portion 240 can at least partially enclose the first handle loop of the Kelly clamp tool. A second case portion 242, separate from the first case 240 portion, can enclose the second handle loop of the Kelly clamp. One or more ratchet ends 244 of the Kelly clamp can protrude from the case 202 through one or more ratchet channels 246 in the case 202. As such, a user can still open and close the Kelly clamp tool and lock the Kelly clamp tool with the ratchet ends 244 when the Kelly clamp tool is at least partially enclosed by the case 202. The case 202 can be manufactured using 3D printed techniques, injection molding techniques, and the like.

In some examples, the one or more force-sensing resistor(s) 104 (and/or other types of force sensors) are mounted or coupled to the case 202. The one or more force-sensing resistor(s) 104 can be disposed at the terminating end 226 or at an intermediate (e.g., middle) portion of the case 202 so that the one or more force-sensing resistor(s) 104 contact a finger of the trainee when the insertion tool 204 is pushed into the training model 130. For instance, the force-sensing resistor(s) 104 can be disposed along a shaft of the insertion device 204 at a location where an index finger contacts the insertion device 204. Additionally, or alternatively, the one or more force-sensing resistor(s) 104 can be located at various other locations in the case 202, such as at the loop handles. The one or more force-sensing resistor(s) 104 can detect pressure or force being applied by a user, such as a hand or finger pressing against the case 202. The chest tube insertion test device 102 can include the one or more force-sensing resistor(s) 104 at a combination of the locations discussed above. In some instances, the chest tube insertion test device 102 receives multiple voltage signals representing an insertion force (e.g., a chest tube insertion force) from multiple force-sensing resistors 104, which can be normalized, aggregated, and/or averaged by the microcontroller 106 to determine an insertion force value corresponding to the multiple voltage signals. In some examples, multiple force-sensing resistors 104 can be disposed on the case 202 to detect whether the trainee is properly gripping the chest tube insertion test device 102 (e.g., based on comparing the detected force to one or more grip-based threshold values). The location of the force-sensing resistor(s) 104 can correspond to whether the chest tube insertion test device 102 is a right-handed device or a left-handed device (e.g., with different finger loop positions and/or same index finger positions). Moreover, the force-sensing resistor(s) 104 can be integrated into the case 202.

FIG. 3 illustrates an example system 300 including the chest tube insertion test device 102 and various components of the chest tube insertion test device 102. FIG. 3 illustrates the various components separately from each other and/or separate from the case 202 for ease of explanation, however, it is to be understood that any of the various components of the chest tube insertion test device 102 can be combined or integrated together with the case 202 and/or the housing 502 illustrated in FIGS. 5A and 5B.

In some examples the chest tube insertion test device 102 includes the one or more feedback output device(s) 122, such as the audio speaker 124 and/or the visual display 126. The audio speaker 124 can be any type of speaker for converting the feedback selected or generated by the feedback generator 120 (e.g., the audio files 116) into sounds. For instance, the audio speaker 124 can be a miniature dynamic speaker with a cone diameter of one inch or less. The audio speaker 124 can be a printed circuit board (PCB) mountable audio speaker and/or a 2-watt or 8-ohm speaker. The visual display 126 can be any type of display for converting the feedback selected or generated by the feedback generator 120 (e.g., the visual data files 118) into a visual output or visual cue. The visual display 126 can be an electronic display such as a liquid crystal display (LCD), a light-emitting diode (LED) display, an organic LED (OLED) display, an active matrix display, an LED backlit LCD, a thin-film transistor (TFT) LCD, an electroluminescent display (ELD), a plasma display, a quantum dot (QLED) display, a segment display, or combinations thereof. In some instances, the visual display 126 is a 4D Systems LCD display that is 5 inches wide with an 800×480 resolution and a 5-volt input.

In some instances, the audio speaker 124 and/or the visual display 126 are formed (e.g., integrated) into the chest tube insertion test device 102. In other examples, one or more of the audio speaker 124 or the visual display 126 can be separate from the chest tube insertion test device 102. For instance, the audio speaker 124 and/or the visual display 126 can be a part of a remote computing system in communication with the chest tube insertion test device 102 (e.g., via a wireless communication interface or a wired communication interface). The remote computing system can be used for remote or virtual training so the chest tube insertion test device 102 and/or an instructor can supervise and provide feedback to the trainee remotely (e.g., from a different building, city, country, etc.)

In some examples, the chest tube insertion test device 102 includes a power supply 302 to provide power to the microcontroller 106 and/or other components of the chest tube insertion test device 102. The power supply 302 can be integrated and/or coupled to the case 202 and/or the housing 502 (FIG. 5). The power supply 302 can be a battery, such as a 9-volt battery, a powerboost module (e.g., including 3.7-volt or 5-volt lithium ion or lithium polymer battery) or combinations thereof. In some instances, the power supply 302 can include an alternating current power source in connection with a rectifier.

FIG. 4 illustrates an example system 400 including the one or more force-sensing resistor(s) 104 of the chest tube insertion test device 102. FIG. 4 illustrates a schematic diagram of internal circuitry connecting the various components of the chest tube insertion test device 102. The system 400 can have a plurality of force sensing resistors 104 such as a first force-sensing resistor, a second force-sensing resistor, and a third force-sensing resistor. The plurality of force-sensing resistors 104 can be electrically wired in parallel, for instance, between a power switch 402 and the power supply 302. Signals from the force-sensing resistors 104 can be evaluated separately and/or additively. For instance, the first force-sensing resistor can couple to a first input port of the microcontroller 106, the second force-sensing resistor can couple to a second input port of the microcontroller 106, and the third force-sensing resistor can couple to a third input port of the microcontroller 106. As such, the microcontroller 106 can receive multiple voltage signals responsive to a force exerted on the plurality of force-sensing resistors 104 and can normalize, aggregate, and/or calculate an average for the multiple voltage signals using the input signal conditioner 112. As such, the system microcontroller determines a chest tube insertion force value corresponding to the force exerted on the plurality of force-sensing resistors 104. The force-sensing resistor(s) 104 can be Ohmite sensors for detecting between 20 grams and 5 kilograms of applied force. The force-sensing resistor(s) 104 can be between 7 millimeters (mm) and 18 mm in size.

In some examples, the audio speaker 124, the visual display 126, and/or other feedback output devices 122 are electrically connected to the microcontroller 106 at one or more output ports of the microcontroller 106 (e.g., and a ground port of the microcontroller 106). For instance, the audio speaker 124 can be electrically wired to a first output port of the microcontroller 106, the visual display 126 can be electrically wired to second output port of the microcontroller 106, and the haptic feedback device can be electrically wired to a third output port of the microcontroller 106.

FIGS. 5A and 5B illustrate an example system 500 including a housing 502 of the chest tube insertion test device 102. FIG. 5A illustrates the housing 502 with a substantially rectangular shape, and FIG. 5B further illustrates how the various components of the chest tube insertion test device 102 can be arranged in or on the housing 502.

In some examples, the housing 502 is formed of a rigid material such as polycarbonate or another rigid material (e.g., metal, plastic, ceramics, composites thereof, etc.). The housing 502 can have one or more external surfaces for mounting or coupling to various components of the chest tube insertion test device 102. For instance, the feedback output device(s) 122, such as the audio speaker 124 and/or the visual display 126 can couple to (or at least extend through) the external surfaces of the housing 502. The power supply 302 and the microcontroller 106 can be mounted or coupled to the housing 502 in an interior space (or at least partially within the interior space) of the housing 502. For instance, the power supply 302 and/or the microcontroller 106 can couple to an inner mounting surface of the housing 502. In some instances, the power switch 402 is disposed inside the housing 502 such that a portion of the housing is removable to gain access to the power switch 402. Alternatively, the power switch 402 can be disposed at the external surfaces of the housing 502.

In some examples, the housing 502 can be a substantially box-shaped enclosure with a width dimension between four inches and six inches, a length dimension between seven inches and nine inches, and a height dimension between nine inches and 11 inches. Alternatively, the housing 502 can be other shapes, such as a circular shape or an irregular shape with contours that conform to match the shapes of the various components contained within and/or mounted to the housing 502.

As illustrated in FIGS. 2A, 2B, 5A, and 5B, the housing 502 can be a separate component from the case 202 and can attach to the case 202 to form the chest tube insertion test device 102. However, in some instances, the housing 502 and the case 202 are combined, integrated, or at least partially combined or integrated in various combinations. For instance, any of the components shown housed in the housing 502 (e.g., the power supply 302, the microcontroller 106, internal circuitry, etc.) can be contained in the case 202. Any of the components shown mounted to the external surfaces of the housing 502 can be mounted to external surfaces of the case 202. Similarly, one or more of the force-sensing resistor(s) 104 can be disposed at the housing 502. The housing 502 and the case 202 may be formed of separate bodies coupled together, or the housing 502 and the case 202 may be formed (e.g., manufactured) integrally as a single body.

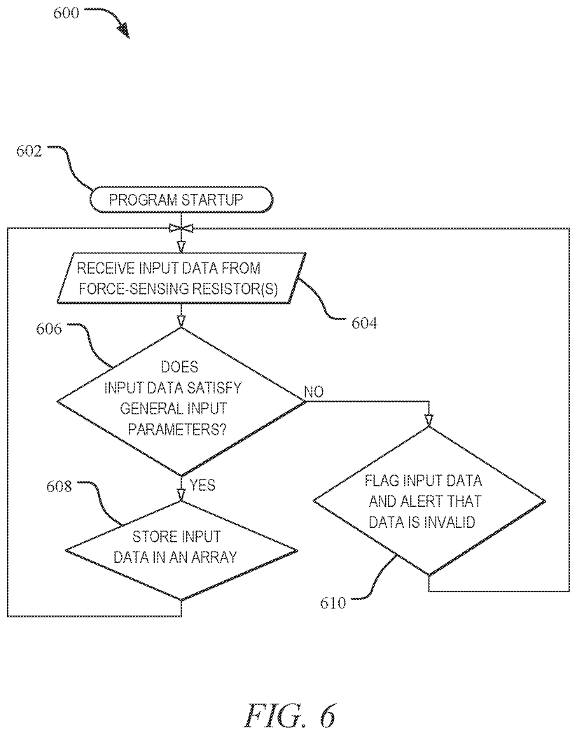

FIGS. 6-8 illustrate example operations performed by the chest tube insertion test device 102 to generate the feedback representing the applied chest tube insertion force. The blocks illustrated in FIGS. 6-8 can represent algorithmic steps or operations of methods performed by the chest tube insertion test device 102 (e.g., by the processor 110 of the microcontroller 106 executing software stored on the one or more memory device(s) 108). FIGS. 6-8 can represent methods implemented by any of the systems 100-500.

For instance, FIG. 6 illustrates a flow chart of an example method 600 for testing and configuring the chest tube insertion test device 102. At operation 602, the method 600 initiates a program start-up. For instance, the power switch 402 can be actuated to provide power to the microcontroller 106 and/or a software application executing on the microcontroller 106 (e.g., Workshop4 IDE) can boot up. At operation 604, the method 600 receives input data from the force-sensing resistor(s) 104. For instance, the force-sensing resistor(s) 104 can generate one or more voltage signals representing a force being applied to the force-sensing resistor(s) 104. At operation 606, the method 600 determines whether the input data satisfies general input parameters. For instance, the memory device(s) 108 can store one or more configuration threshold values representing the general input parameters. Upon receiving the input data, the microcontroller 106 (e.g., the input signal conditioner 112 component of the microcontroller 106) can compare the input data to the configuration threshold values to determine if the input data is valid data or if an error is occurring in the data collection process. For instance, the one or more configuration threshold values can indicate a force range between 20 grams and 5 kilograms. If, at operation 606, the method 600 determines that the input data satisfies the general input parameters (e.g., the input data is within the force range), the method 600 can proceed to operation 608. At operation 608, the method 600 stores the input data in a data array (e.g., at the one or more memory devices 108). The input data stored at operation 608 can be further analyzed by the feedback generator 120, as discussed in greater detail below. If, at operation 606, the method 600 determines that the input data does not satisfy the general input parameters (e.g., the input data is outside the force range), the method 600 can proceed to operation 610. At operation 610, the system flags the input data as invalid input data (e.g., stored at the memory device(s) 108 with an “invalid” association) and generates an invalid data alert indicating that the input data is invalid input data. For instance, the invalid data alert can be one of the audio files 116 and/or one of the visual data files 118 outputted by the audio speaker 124 and/or the visual display 126. The invalid data alert can include an error message and/or an instruction for how to operate the chest tube insertion test device 102.

FIG. 7 illustrates a flow chart of an example method 700 for generating feedback using the chest tube insertion test device 102. The method 700 can be performed to provide continuous or real-time feedback as visual or auditory feedback, based on how hard or soft the trainee is pushing the insertion tool 204 into the training model 130. At operation 702, the method 700 initiates a program start-up. For instance, the power switch 402 can be actuated to provide power to the microcontroller 106 and/or a software application executing on the microcontroller 106 (e.g., Workshop4 IDE) can boot up. At operation 704, the method 700 receives input data from the force-sensing resistor(s) 104. For instance, the force-sensing resistor(s) 104 can generate one or more voltage signals generated by and representing a force being applied to the force-sensing resistor(s) 104 as the insertion tool 204 and the chest tube insertion test device 102 are pushed into the training model 130. The chest tube insertion force can be generated by a hand of the trainee squeezing or pushing the chest tube insertion test device 102 and the insertion tool 204. The chest tube insertion force can be generated by the trainee pushing the insertion tool 204 into an inner pad of the training model 130 such that the inner pad contacts and presses against the force-sensing resistor(s) 104. At operation 706, the system compares the input data (e.g., the voltage signals normalized by the input signal conditioner 112) to theoretical values which can be obtained during a testing or configuration process (e.g., the predetermined threshold values 114 stored in the memory device(s) 108). The predetermined threshold values 114 can include a first predetermined threshold value as a lower end of a range of values indicating an expected force range. The first predetermined threshold value can be a value greater than zero. The predetermined threshold values 114 can include a second predetermined threshold value greater than the first predetermined threshold value, which can be an upper end of the range of values indicating the expected force range. Furthermore, the predetermined threshold values 114 can include a force rate of change threshold value for indicating whether the insertion force has a rate of change indicating that the insertion tool 204 has reached an inner pad of the training model 130, as discussed in greater detail below.

In some instances, at operation 706, the method 700 determines that the chest tube insertion force (as represented by the voltage signal(s)) is within the expected force range indicated by the first predetermined threshold value and the second predetermined threshold value. In response, the method 700 can proceed to operation 708. At operation 708, the method 700 outputs feedback in the form of a visual cue and/or an audio cue (e.g., by executing the audio file 116 or the visual data file 118). The feedback outputted at operation 708 can include an alert or instruction indicating that the chest tube insertion force is within the expected force range and/or the trainee should continue pushing the insertion tool 204 with a same or similar chest tube insertion force.

In some instances, at operation 706, the method 700 determines that the chest tube insertion force (e.g., a first chest tube insertion force) is less than the expected force range (e.g., less than the first predetermined threshold value). In response, the method 700 can proceed to operation 710. At operation 710, the method 700 outputs the feedback as one or more visual cues and/or audio cues. The feedback outputted at operation 710 can include an alert or an instruction indicating that the first chest tube insertion force is less than or outside of the expected force range and/or the trainee should push the insertion tool 204 with a second chest tube insertion force that is greater than the first chest tube insertion force. The feedback generator 120 can calculate a difference between the first chest tube insertion force and one of the predetermined threshold values 114 (e.g., the first predetermined threshold value). Accordingly, the feedback can include an indication of this difference to inform the trainee how much more force should be applied to arrive at the second chest tube insertion force.

In some instances, at operation 706, the method 700 determines that the first chest tube insertion force is greater than the expected force range (e.g., greater than the second predetermined threshold value). In response, the method 700 can proceed to operation 712. At operation 712, the method 700 can determine whether the first chest tube insertion force is greater or less than a third predetermined threshold value. The third predetermined threshold value is greater than the second predetermined threshold value and, with the second predetermined threshold value, defines a second force range above the expected force range. In other words, at operation 712, the method 700 determines the degree to which the first chest tube insertion force exceeds the expected force range, and determines which feedback to output based on amount by which the first chest tube insertion force exceeds the expected force range (e.g., as indicated by comparing the first chest tube insertion force to the third predetermined threshold value and/or determining whether the first chest tube insertion force is within the second force range).

For instance, at operation 712, the method 700 can determine that the first chest tube insertion force is less than the third predetermined threshold value (e.g., within the second force range and greater than the expected force range). In response, the method 700 proceeds to operation 714. At operation 714, the method 700 outputs the feedback including an alert or an instruction indicating that the first chest tube insertion force is greater than or outside of the expected force range and the trainee should push the insertion tool 204 with a second chest tube insertion force that is less than the first chest tube insertion force. The feedback generator 120 can calculate a difference between the first chest tube insertion force and one of the predetermined threshold values 114 (e.g., the second predetermined threshold value). Accordingly, the feedback can include an indication of this difference to inform the trainee how much less force should be applied to arrive at the second chest tube insertion force.

Alternatively, at operation 712, the method 700 can determine that the first chest tube insertion force is greater than the third predetermined threshold value (e.g., greater than the second force range in addition to being greater than the expected force range). In response, the method 700 proceeds to operation 716. At operation 716, the method 700 generates and/or outputs feedback instructing the trainee to stop pushing the insertion tool 204 and/or to inspect an insertion area of the training model 130. For instance, the method 700 can determine that the chest tube insertion force is significantly greater than the expected force range (due to being greater than the second range as well as the expected force range) and, as such, determine that the insertion tool 204 may be improperly positioned or pushing against a bone. The feedback generator 120 can generate and/or cause an instruction or alert to be outputted conveying this information to the trainee. For instance, the feedback can indicate that the insertion tool 204 is likely being pushed against a bone and instruct the trainee to inspect the insertion area to determine if the insertion tool 204 is being pushed against the bone.

In some examples, at operation 718, the method 700 can determine that the input data represents a sudden change in force. For instance, one of the predetermined threshold values 114 can be the force rate of change threshold value indicating a threshold rate of change for the chest tube insertion force. If, at operation 718, the input data indicates that the chest tube insertion force has a rate of change less than the rate of change represented by the force rate of change threshold value, the method 700 proceeds to operation 720. If, alternatively, the input data indicates that the chest tube insertion force has a rate of change greater than the rate of change represented by the force rate of change threshold value, the method 700 proceeds to operation 722. The threshold force rate of change value can represent a negative rate of change. In other words, the method 700 can use the threshold force rate of change value to determine when the chest tube insertion force drops suddenly (e.g., changes in a negative direction at a rate greater than the threshold rate). Operation 718 can be performed subsequently to operation 706 or, additionally or alternatively, operation 718 can be performed in parallel with operation 706. The force rate of change can be caused by the insertion tool 204 passing through an inner pad of the training model 130.

In some examples, the method 700 proceeds from operation 718 to operation 720 in response to the rate of change of the chest tube insertion force being less than the predetermined threshold value 114. At operation 720, the system determines to continue the training procedure by looping back to operation 704, such that the method 700 again receives the input data from the force-sensing resistor(s) 104, normalizes the input data with the input signal conditioner 112, and/or outputs feedback with the feedback generator 120.

In some examples, the method 700 proceeds from operation 718 to operation 722 in response to the rate of change (e.g., an absolute rate of change) of the chest tube insertion force being greater than the predetermined threshold value 114. At operation 722, the method 700 generates and/or outputs an alert or an instruction (e.g., feedback via the feedback generator 120 selecting the audio file 116 or the visual data file 118) informing the trainee that the training procedure is complete and/or that the insertion tool 204 has reached a terminal or final location. For instance, the feedback can indicate that the chest tube insertion test device 102 and the insertion tool 204 have passed through the inner pad (e.g., a training chest wall pad) of the training model 130 representing entering the thoracic cavity. As a result, the method 700 can proceed to operation 724. At operation 724, the method 700 completes the training procedure. For instance, the method 700 can stop receiving input data from the force-sensing resistor(s) 104 and/or stop providing power from the power supply 302 to the microcontroller.

In some example, the method 700 can perform the operations 704-720 repeatedly or iteratively. After performing many of the operations illustrated in FIG. 7, the method 700 can subsequently perform operation 704 and repeat the method illustrated in FIG. 7 multiple times. For instance, the method 700 can perform operation 704 and receive input data from the force-sensing resistor(s) in response to operation 708, operation 710, operation 714, operation 716, and/or operation 720. As such, the method 700 provides continuous, real-time feedback to the trainee indicating their progress towards completing the chest tube insertion procedure properly with the appropriate amount of insertion force.

In some examples, generating the alerts, instructions, or audio cues discussed herein includes selecting and executing, with the feedback generator 120, one or more audio files 116. For instance, the instruction to continue pushing the insertion tool 204 with the first chest tube insertion force can include a first audio message (e.g., generated by outputting a first audio file of the audio files 116) with a first set of words or phrases to that effect (e.g., “continue pushing with the same force,” “keep going,” etc.). The instruction to push the insertion tool 204 with the second chest tube insertion force that is greater than the first chest tube insertion force can include a second audio message (e.g., generated by outputting a second audio file of the audio files 116) with a second set of words or phrases (e.g. “push harder,” “increase the insertion force,” etc.). The instruction to push the insertion tool 204 with the second chest tube insertion that is less than the first chest tube insertion force can include a third audio message (e.g., generated by outputting a third audio file of the audio files 116) with a third set of words or phrases (e.g., “push softer,” “push with less force,” “decrease the insertion force,” etc.). The instruction to stop applying the first chest tube insertion force and/or to inspect the insertion area (e.g., to determine if the insertion tool is pressing against bone) can include a fourth audio message (e.g., generated by outputting a fourth audio file of the audio files 116) with a fourth set of words or phrases (e.g., “stop,” “stop pushing,” “stop applying force,” “pause,” “inspect the insertion area,” and/or “check for bone obstruction,” etc.). The instruction that the training operation is complete can include a fifth audio message (e.g., generated by outputting a fifth audio file of the audio files 116) with a fifth set of words or phrases (e.g., “training complete,” “stop,” “stop pushing,” “end,” etc.). In other words, the feedback generator 120 can select and output multiple audio messages or cues providing multiple instructions to the trainee throughout the training procedure. Moreover, the audio cues may include non-verbal audio cues such as beeps or tones. For instance, the audio cues can include a tone having a pitch that has a lower frequency to indicate proper force, and increases to a higher frequency to indicate that the force is greater than the predetermined threshold value.

In some examples, generating the alerts, instructions, or visual cues discussed herein includes selecting and executing, with the feedback generator 120, one or more visual data files 118. For instance, outputting the visual cues can include displaying one or more images, videos, animations, diagrams, icons, color-codings, text words or phrases, or other visual messages. Visual cues including text words or phrases can present any of the words or phrases discussed above regarding audio cues. For instance, the feedback generator 120 can select, generate, or output the instruction to continue pushing the insertion tool 204 with the first chest tube insertion force as a first visual cue (e.g., generated by outputting a first visual data file of the visual data files 118) indicating a first visual message (e.g., “continue pushing with the same force,” “keep going,” etc.). The instruction to push the insertion tool 204 with the second chest tube insertion force that is greater than the first chest tube insertion force can include a second visual cue (e.g., generated by outputting a second visual data file of the visual data files 118) indicating a second visual message (e.g. “push harder,” “increase the insertion force,” etc.). The instruction to push the insertion tool 204 with the second chest tube insertion that is less than the first chest tube insertion force can include a third visual cue (e.g., generated by outputting a third visual data file of the visual data files 118) with a third visual message (e.g., “push softer,” “push with less force,” “decrease the insertion force,” etc.). The instruction to stop applying the first chest tube insertion force and/or to inspect the insertion area (e.g., to determine if the insertion tool is pressing against bone) can include a fourth visual cue (e.g., generated by outputting a fourth visual data file of the visual data files 118) with a fourth visual message (e.g., “stop,” “stop pushing,” “stop applying force,” “pause,” “inspect the insertion area,” and/or “check for bone obstruction,” etc.) The instruction that the training operation is complete can include a fifth visual cue (e.g., generated by outputting a fifth visual data file of the visual data files 118) with a visual message (e.g., “training complete,” “stop,” “stop pushing,” “end,” etc.). In other words, the feedback generator 120 can select and output multiple visual cues or messages providing multiple instructions to the trainee throughout the training procedure. Moreover, any of the visual cues discussed herein can include a video showing the insertion tool 204 being pushed into the training model and/or an arrow, number, color, or diagram representing the first chest tube insertion force, the second chest tube insertion force, an expected chest tube insertion force (e.g., the expected force range), or combinations thereof. The feedback generator 120 can output one or more visual cues in addition to or alternately to outputting the one or more audio cues (e.g., simultaneously or subsequently).

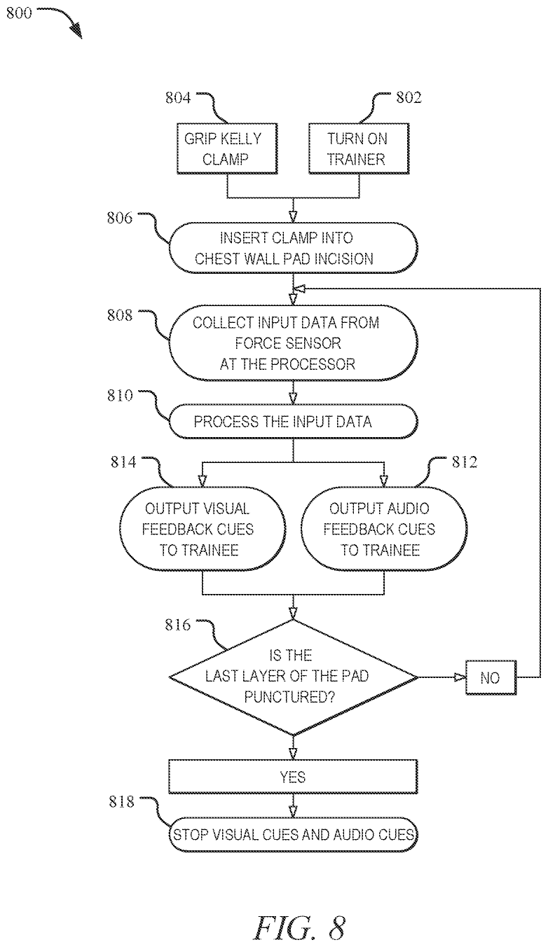

FIG. 8 illustrates a flow chart of an example method 800 for generating feedback using the chest tube insertion test device 102. At operation 802, the method 800 turns on the chest tube insertion test device 102 (e.g., by actuating the power switch 402 and/or providing power to the microcontroller 106). At operation 804, the method 800 grips (e.g., using a hand of a trainee) the insertion tool 204, such as the Kelly clamp tool. At operation 806, the method 800 inserts the insertion tool 204 into a chest wall pad incision (e.g., the inner pad of the training model 130). At operation 808, the method 800 collects the input data from a force sensor (e.g., the force-sensing resistor(s) 104) at a processor (e.g., the processor 110 of the microcontroller 106). At operation 810, the method 800 processes the input data (e.g., normalizes the voltage signals with the input signal conditioner 112 and/or determines which feedback to output with the feedback generator 120). At operation 812, the method 800 outputs audio feedback cues to the trainee (e.g., by outputting one or more audio files 116 with the audio speaker 124). At operation 814, the method 800 outputs visual feedback cues to the trainee (e.g., by outputting one or more visual data files 118 with the visual display 126). In some instances, operations 812 and 814 occur simultaneously. Following operations 812 and/or 814, the method 800 proceeds to operation 816 to determine whether a last layer of the inner pad of the training model 130 is punctured (e.g., by comparing the rate of change of the insertion force to the predetermined threshold value 114). If, at operation 816, the method 800 determines that the last layer of the inner pad is not punctured, the method 800 proceeds to operation 808 and continues collecting the input data so that the method 800 can repeat in an iterative manner. If, at operation 816, the method 800 determines that the last layer of the inner pad is punctured, the method 800 proceeds to operation 818. At operation 818, the method 800 stops providing visual cues and/or audio cues, thereby indicating that a training procedure is complete).

It is to be understood that the specific order or hierarchy of steps in the methods depicted in FIGS. 6-8 (and other methods disclosed herein) are instances of example approaches and can be rearranged while remaining within the disclosed subject matter. For instance, any of the operations depicted in FIGS. 6-8 can be omitted, repeated, performed in parallel, performed in a different order, and/or combined with any other of the operations depicted in FIGS. 6-8. Moreover, any of the systems or methods illustrated in FIGS. 1-8 can be combined together and/or form at least a portion of the system 100.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. An apparatus to provide feedback for a chest tube insertion, the apparatus comprising:
a case coupled to an insertion tool;
one or more force-sensing resistors coupled to the case;
one or more feedback output devices;
a microcontroller communicatively coupled to the one or more force-sensing resistors and the one or more feedback output devices; and
one or more memory devices configured to store instructions that, when executed by the microcontroller, cause the apparatus to:
generate, using the one or more force-sensing resistors, an input signal representing a chest tube insertion force,
determine whether the chest tube insertion force exceeds a predetermined threshold value, and
output, using the one or more feedback output devices, the feedback indicating whether the chest tube insertion force exceeds the predetermined threshold value.

2. The apparatus of claim 1, wherein the one or more force-sensing resistors are a plurality of force-sensing resistors electrically wired in parallel.

3. The apparatus of claim 1,
wherein,
the predetermined threshold value corresponds to an expected force range, and
the feedback includes an indication of whether the chest tube insertion force is within the expected force range.

4. The apparatus of claim 1,
wherein,
the instructions, when executed by the microcontroller, further cause the apparatus to determine the chest tube insertion force is less than the predetermined threshold value, and
the feedback includes an alert instructing a user to push the insertion tool with more force.

5. The apparatus of claim 1,
wherein,
the instructions, when executed by the microcontroller, further cause the apparatus to determine the chest tube insertion force exceeds the predetermined threshold value, and
the feedback includes a first alert instructing a user to push the insertion tool with less force or a second alert instructing the user to inspect an insertion area.

6. The apparatus of claim 5,
wherein,
the one or more feedback output devices include at least one of a display or an audio speaker,
the first alert includes at least one of a first visual cue presented on the display or a first audio file outputted by the audio speaker, and
the second alert includes at least one of a second visual cue presented on the display or a second audio file outputted by the audio speaker.

7. The apparatus of claim 1, wherein the chest tube insertion force corresponds to between zero kilograms and five kilograms.

8. The apparatus of claim 1,
wherein,
the case is removably secured to the insertion tool, and
the case, when secured to the insertion tool, at least partially encloses a portion of the insertion tool.

9. The apparatus of claim 8,
wherein,
the insertion tool is a Kelly clamp tool,
the portion of the insertion tool at least partially enclosed by the case is a handle portion of the Kelly clamp tool.

10. The apparatus of claim 1, further comprising:
a housing coupled to the case,
wherein,
the microcontroller is at least partially contained in the housing, and
the one or more feedback output devices are coupled to the housing.

11. The apparatus of claim 10, further comprising:
a power supply at least partially contained in the housing, the power supply providing power to the microcontroller.

12. A system to provide feedback for a chest tube insertion, the system comprising:
a force sensor coupled to an insertion tool;
a feedback output device;
a microcontroller communicatively coupled to the force sensor and the feedback output device; and
one or more memory devices storing instructions that, when executed by the microcontroller, cause the system to:
receive, using the force sensor, an input signal representing a chest tube insertion force, and
generate, using the feedback output device, an output indicating that:
the chest tube insertion force is below a predetermined threshold range,
the chest tube insertion force is within the predetermined threshold range, or
the chest tube insertion force is greater than the predetermined threshold range.

13. The system of claim 12,
wherein,
the feedback output device includes an audio speaker, and
generating the output includes playing an audio file with the audio speaker.

14. The system of claim 12,
wherein,
the feedback output device includes a display, and
generating the output includes presenting one or more visual cues on the display.

15. The system of claim 12,
wherein,
the chest tube insertion force is a first chest tube insertion force, and
the output further includes an instruction to:
push the insertion tool with a second chest tube insertion force being less than the first chest tube insertion force,
push the insertion tool with the second chest tube insertion force being greater than the first chest tube insertion force, or
continue pushing the insertion tool with the first chest tube insertion force.

16. A method to provide feedback for a chest tube insertion, the method comprising:
generating, with a force sensor coupled to an insertion tool, an input signal representing a chest tube insertion force for the insertion tool;
receiving the input signal at a microcontroller communicatively coupled to the force sensor;
determining whether the chest tube insertion force exceeds a predetermined threshold value; and
outputting, at one or more feedback output devices, the feedback indicating whether the chest tube insertion force exceeds the predetermined threshold value.

17. The method of claim 16, wherein the force sensor includes one or more force-sensing resistors integrated into a case at least partially enclosing the insertion tool.

18. The method of claim 16,
wherein,
generating the input signal includes:
inserting the insertion tool into a training model, and
causing an inner pad of the training model to be pressed against the force sensor.

19. The method of claim 18, further comprising:
detecting, with the force sensor, a change in force caused by the insertion tool passing through the inner pad of the training model.

20. The method of claim 19, wherein the feedback further indicates, in response to the change in force, a training procedure is complete.
